(19) United States
(12) Patent Application Publication
(10) Pub. No.: US 2014/0274779 A1
(43) Pub. Date: Sep. 18, 2014

(54) MULTIPLEX ALLELE DETECTION

(71) Applicant: Abbott Molecular Inc., Des Plaines, IL (US)

(72) Inventors: Shihai Huang, Lincolnshire, IL (US); Hong Su, Evanston, IL (US)

(73) Assignee: Abbott Molecular Inc., Des Plaines, IL (US)

(21) Appl. No.: 14/210,618

(22) Filed: Mar. 14, 2014

Related U.S. Application Data
(60) Provisional application No. 61/792,202, filed on Mar. 15, 2013.

(51) Int. Cl. C12Q 1/68

(52) U.S. Cl. CPC C12Q 1/6818; C12Q 1/6827; C12Q 1/6886; C12Q 1/6858
USPC 506/9; 435/6.12; 435/6.11; 506/16

(57) ABSTRACT

Provided herein is technology relating to nucleic acid detection and particularly, but not exclusively, to methods and compositions for the simultaneous detection of multiple nucleic acids.

This application claims priority to U.S. provisional patent application Ser. No. 61/792,202, filed Mar. 15, 2013, which is incorporated herein by reference in its entirety.

### FIELD OF INVENTION

Provided herein is technology relating to nucleic acid detection and particularly, but not exclusively, to methods and compositions for the simultaneous detection of multiple nucleic acids.

### BACKGROUND

Detection of single nucleotide polymorphisms (SNPs) finds use in biomedical fields such as human genetics, cancer diagnostics, pharmacogenetics, and microbial genotyping. In general, many conventional technologies detect SNPs with allele-specific primers that are designed to have a sequence that efficiently extends a specific SNP template, but not a wild-type template or other non-targeted SNP sequences. As a result of their specificity, such allele-specific primers are often preferred for SNP detection relative to other technologies such as, e.g., allele-specific probes. However, PCR and real-time PCR methods using allele specific primers are limited in their multiplexing capabilities because detecting an amplified product is either based on a probe binding to a consensus sequence or based on technologies that do not have sufficient capability to differentiate between small nucleotide variations such as individual SNPs.

Some conventional solutions use allele-specific primers comprising SNP-detecting nucleotides at their 3′ ends and primer-specific hybridization tags (U.S. Pat. No. 6,794,133). However, this technology depends on solid phase technology and has problems with primer specificity that compromise its utility for real-time PCR. Other conventional haplotyping solutions provide multiplex detection with allele-specific primers comprising SNP-detecting nucleotides at their 3′ ends, but detection of SNPs is accomplished by size and not by real-time PCR fluorescent signals (Intl Pat. Pub. No. WO 2008143367). Finally, some extant conventional technologies use universal primer sequences for multiplex analysis, but detection is performed by electrophoresis and does not provide for multiplex real-time PCR (U.S. Pat. Nos. 6,207,372; 5,882,856). Accordingly, there is a need in the biomedical field for multiplexed PCR detection of multiple SNPs with allele-specific primers.

### SUMMARY

Provided herein is technology relating to a multiplexed detection of nucleic acid sequences using allele-specific primers. For example, embodiments of methods are provided for the simultaneous detection of multiple SNPs in one detection reaction (e.g., a PCR, e.g., a real-time PCR). In some embodiments, the technology is referred to herein as multiplex allele-specific priming detection (Masp) and as FRET-mediated multiplex allele specific priming detection (F-Masp).

In some embodiments, and as the technology finds use in some applications, F-Masp provides one or more advantages relative to Masp in its use of a probe that, in some embodiments, provides additional specificity to the technology when desired. For example, F-Masp reduces or eliminates in some assays interference from non-specific signals, e.g., from non-specific priming, the formation of primer dimers, and/or the presence of non-targeted regions that contain the same or similar target sequences (e.g., pseudo-genes).

This technology provided herein allows the detection, identification, and/or reporting of multiple SNPs or genotypes from one homogeneous amplification reaction. As such, the technology provides clinicians and patients with information more efficiently, improves assay workflow, and provides a needed technology.

This technology, in some embodiments, provides a method for detecting a nucleic acid in a sample, the method comprising contacting a sample comprising a nucleic acid with a primer in a quenched state; and detecting a signal from the primer in a detectable state if the primer hybridizes to the nucleic acid and generates amplification products, wherein the nucleic acid is detected in the sample when the signal is detected. In some embodiments, the primer in the quenched state comprises a double-stranded duplex region. In some embodiments, the signal is fluorescence. In some embodiments, the primer comprises a fluorophore and the fluorophore is quenched by a quencher when the primer is in the quenched state. The technology is not limited in the fluorescent moieties, quencher moieties, and FRET pairs that are linked to oligonucleotides. For example, some embodiments provide a method wherein the fluorophore is FAM, TET, JOE, HEX, TAMRA, ROX, Cy3, Cy5, or Cy5.5; and the quencher is BHQ-1, BHQ-2, or BHQ-3.

In some embodiments, the primer in the detectable state is hybridized to the nucleic acid; in some embodiments, the primer in the quenched state is incorporated into a strand of a nucleic acid by a polymerase. In some embodiments, the primer in the quenched state is incorporated into a strand of nucleic acid by a polymerase and the primer retains the double-stranded duplex region; then, in some embodiments, the primer is in the detectable state upon loss of the double-stranded duplex region, e.g., upon synthesis of a complementary strand that displaces the double-stranded duplex region.

Primers according to the technology are provided in several forms. For example, in some embodiments the primer in the quenched state consists of one oligonucleotide comprising a fluorophore and a quencher. In some embodiments, the primer in the quenched state consists of a first oligonucleotide comprising a fluorophore and a second oligonucleotide comprising a quencher, wherein the first oligonucleotide is hybridized to the second oligonucleotide. In some embodiments, the primer in the unexcited state consists of a first oligonucleotide comprising a fluorophore and a second oligonucleotide, wherein the first oligonucleotide is hybridized to the second oligonucleotide.

In some embodiments, the primers of the technology are incorporated into a nucleic acid by a polymerase (e.g., during PCR) or another synthetic process. In some embodiments, the incorporated primer is in a detectable state, in a quenched state, or in an unexcited state. Accordingly, in some embodiments an amplicon comprises the primer in the detectable state. Primers according to the technology are provided in several forms. For example, in some embodiments, the primer is a stem-loop primer or a double-stranded linear primer. In some embodiments, the primer is an allele-specific primer.

In some embodiments, the technology further comprises extending the primer in the detectable state with a polymerase and a nucleotide and in some embodiments the technology further comprises performing a polymerase chain reaction, e.g., in some embodiments the polymerase chain reaction is a real-time polymerase chain reaction.

In some embodiments the methods are multiplex methods for detecting more than one nucleic acid, e.g., more than one allele, SNP, gene, mutation, etc. Accordingly, in some embodiments the methods comprise contacting the sample comprising a second nucleic acid with a second primer in a quenched state; and detecting a second signal from the second primer in a detectable state if the second primer hybridizes to the second nucleic acid and generates amplification products, wherein the second nucleic acid is detected in the sample when the second signal is detected.

In some embodiments the technology comprises use of a primer and a probe. For instance, in some embodiments are provided a method for detecting a nucleic acid in a sample, the method comprising contacting a sample comprising a nucleic acid with a primer and a probe in a quenched state or in an unexcited state; and detecting a signal from the primer if the probe hybridizes to the nucleic acid, wherein the nucleic acid is detected in the sample when the signal is detected. In some embodiments, the probe comprises a double-stranded duplex region. In some embodiments, the signal is fluorescence. In some embodiments, the probe comprises a fluorophore (e.g., a fluorescence resonance energy transfer donor) and the primer comprises a fluorophore (e.g., a fluorescence resonance energy transfer acceptor).

Probes according to the technology are provided in several forms. For example, in some embodiments the probe in the quenched state consists of one oligonucleotide comprising a fluorophore and a quencher. In some embodiments, the probe in the quenched state consists of a first oligonucleotide comprising a fluorophore and a second oligonucleotide comprising a quencher, wherein the first oligonucleotide is hybridized to the second oligonucleotide. In some embodiments, the probe in the unexcited state consists of a first oligonucleotide comprising a fluorophore and a second oligonucleotide, wherein the first oligonucleotide is hybridized to the second oligonucleotide. In some embodiments, the probe is modified (e.g., on its 3′ end) so that it does not provide a substrate for extension by a polymerase (e.g., a polymerase cannot add a nucleotide to the probe). In some embodiments the probe is a stem-loop probe or a double-stranded linear probe as described herein. The technology finds use in detecting alleles, SNPs, mutations, etc. and in some embodiments the primer is an allele-specific primer.

In some embodiments, the probe fluorophore is quenched by a quencher when the probe is in the quenched state. The technology is not limited in the fluorophores and quenchers that are linked to oligonucleotides of the technology. For example, in some embodiments the fluorophore is FAM, TET, JOE, HEX, TAMRA, ROX, Cy3, Cy5, or Cy5.5; and the quencher is BHQ-1, BHQ-2, or BHQ-3. In some embodiments, the primer comprises a second fluorophore that is a fluorescence resonance energy transfer (FRET) acceptor compatible with the fluorophore that is a fluorescence resonance energy transfer (FRET) donor of the probe.

In some embodiments, the methods comprise extending the primer with a polymerase and a nucleotide, e.g., as in performing a polymerase chain reaction. In some embodiments, the polymerase chain reaction is a real-time polymerase chain reaction.

In some embodiments the methods are multiplex methods for detecting more than one nucleic acid, e.g., more than one allele, SNP, gene, mutation, etc. In some embodiments comprising use of FRET, the same donor moiety is used with two (or more) different acceptor moieties and in some embodiments two (or more) different donor moieties are used with the two (or more) acceptor moieties. Accordingly, in some embodiments the methods further comprise contacting the sample comprising a second nucleic acid with a second primer; and detecting a second signal from the second primer if the probe hybridizes to the second nucleic acid, wherein the second nucleic acid is detected in the sample when the second signal is detected.

The technology provides embodiments of compositions that find use in the detection of one or more alleles, SNPs, genes, mutations, etc. In some embodiments are provided a composition comprising one of the following:
a nucleic acid hybridized to a detectable primer, wherein the detectable primer comprises a fluorophore and a quencher;
a nucleic acid comprising a detectable primer, wherein the detectable primer comprises a fluorophore and a quencher;
a nucleic acid hybridized to a detectable primer, wherein the detectable primer comprises a fluorophore; and a quencher oligonucleotide comprising a quencher;
a nucleic acid comprising a detectable primer, wherein the detectable primer comprises a fluorophore; and a quencher oligonucleotide comprising a quencher;
a nucleic acid comprising a primer and hybridized to a probe, wherein the probe comprises a first fluorophore and a quencher, the primer comprises a second fluorophore, and the first fluorophore and the second fluorophore are a FRET pair; or
a nucleic acid comprising a primer and hybridized to a probe, wherein the probe comprise a first fluorophore, the primer comprises a second fluorophore, and the first fluorophore and the second fluorophore are a FRET pair; and a quencher oligonucleotide comprising a quencher.

In some embodiments the composition is a reaction mixture. In some embodiments the composition further comprises a polymerase and/or a nucleotide (e.g., in a PCR such as a real-time PCR). In some embodiments a fluorophore is FAM, TET, JOE, HEX, TAMRA, ROX, Cy3, Cy5, or Cy5.5; and a quencher is BHQ-1, BHQ-2, or BHQ-3. Some embodiments comprise more than one fluorophore and/or more than one quencher.

Some embodiments provide for multiplex detection of two or more alleles, SNPs, mutations, genes, etc. using combinations of the probes and primers provided herein, e.g., multiple primers (e.g., allele-specific primers). Thus, in some embodiments are provided compositions, methods, systems, and kits for multiplex detection of two (or more) alleles, SNPs, mutations, genes, etc. These embodiments relate to the use of two or more primers as described herein (e.g., two or more allele-specific primers), each labeled with a separately detectable fluorophore. In these embodiments, the quenchers associated with the two or more primers may be the same quenchers or the quenchers associated with the two or more primers may be two or more different quenchers. In embodiments comprising probes and/or use of probes, the probes associated with the two or more primers may be the same probe or the probes associated with the two or more primers may be two or more different probes. Accordingly, the probes used in a multiplex assay may comprise the same sequence or the probes used in a multiplex assay may comprise two or more different sequences (e.g., to hybridize to the same target sequence or to hybridize to two or more different target sequences). In some embodiments, the two or more different sequences to which the probes hybridize may differ by only a single nucleotide. For embodiments of probes that comprise a quencher, each of the two or more different probes may comprise the same quencher or each of the two or more different probes that comprise a quencher may comprise two or more different quenchers. In some embodiments, two or more primers are used for multiplex detection and one probe is used for the detection of the two or more primers. In some embodiments, two or more primers are used for multiplex detection and two or more probes are used for the detection of the two or more primers.

Some embodiments are associated with fluorescence resonance energy transfer (e.g., F-Masp) between a donor fluorophore on a probe and two or more acceptor fluorophores on two or more primers. In these embodiments, the same probe donor fluorophore is used with the two or more primer acceptor fluorophores. Thus, there are at least three fluorophores in the assay, e.g., an acceptor fluorophore on the first primer, a different acceptor fluorophore on the second primer, and a donor fluorophore on the probe that forms a FRET pair with each of the two or more primer fluorophores. In some embodiments, two or more different probes are used for each of the two or more primers. In these embodiments, two or more different probe donor fluorophores are used with the two or more primer acceptor fluorophores. Thus, there are at least four fluorophores in the assay, e.g., an acceptor fluorophore on the first primer, a different acceptor fluorophore on the second primer, a donor fluorophore on the probe associated with the first primer, and different donor fluorophore on the probe associated with the second primer. The fluorophores the primer and probe of each associated primer-probe pair are a FRET pair. In some embodiments, an unhybridized probe comprises a double-stranded duplex region; in some embodiments an unhybridized probe comprises a quencher (e.g., the probe is in an unexcitable state).

Accordingly, in some embodiments (e.g., multiplex embodiments) the compositions further comprise one of the following:
a second nucleic acid hybridized to a second detectable primer, wherein the second detectable primer comprises a second fluorophore and the quencher or a second quencher;
a second nucleic acid comprising a second detectable primer, wherein the second detectable primer comprises a second fluorophore and the quencher or a second quencher;
a second nucleic acid hybridized to a second detectable primer, wherein the second detectable primer comprises a second fluorophore;
a second nucleic acid comprising a second detectable primer, wherein the second detectable primer comprises a second fluorophore;
a second nucleic acid comprising a second primer and hybridized to the probe, wherein the probe comprises the first fluorophore and the quencher, the second primer comprises a third fluorophore, and the first fluorophore and the third fluorophore are a FRET pair; or
a second nucleic acid comprising a second primer and hybridized to the probe, wherein the probe comprise the first fluorophore, the second primer comprises a third fluorophore, and the first fluorophore and the third fluorophore are a FRET pair.

In some embodiments comprising a quencher oligonucleotide, the quencher oligonucleotide is a second quencher oligonucleotide comprising the quencher or a second quencher. Related embodiments provide for the use of a composition as provided herein to detect one or more alleles, to detect one or more single nucleotide polymorphisms, and/or to detect more than one allele in the same sample in a multiplex assay.

Further embodiments provide a kit comprising a detection reagent, wherein the detection reagent comprises one of the following:
a stem-loop primer comprising a fluorophore and a quencher;
a double-stranded linear primer comprising an allele-specific single-stranded primer comprising a fluorophore and a complementary quenching oligonucleotide;
a double-stranded linear primer comprising an allele-specific single-stranded primer comprising a fluorophore and a complementary oligonucleotide;
an allele-specific primer comprising a fluorophore and a double-stranded probe comprising a probe strand comprising a second fluorophore and a quencher oligonucleotide;
an allele-specific primer comprising a fluorophore and a double-stranded probe comprising a probe strand comprising a second fluorophore; or
an allele-specific primer comprising a fluorophore and a stem-loop probe comprising a second fluorophore and a quencher.

In some embodiments, the kit further comprises a control nucleic acid.

In some embodiments, the kits provide for the multiplex detection of more than one allele, SNP, gene, mutation, etc. Thus, in some embodiments the kits further comprise a second detection reagent, wherein the second detection reagent comprises one of the following:
a second stem-loop primer comprising a second fluorophore and the quencher or a second quencher;
a second double-stranded linear primer comprising a second allele-specific single-stranded primer comprising a second fluorophore and the complementary quenching oligonucleotide or a second complementary quenching oligonucleotide; or
a second allele-specific primer comprising a third fluorophore.

Kits include probes, in some embodiments, such as:
a second double-stranded probe comprising a second probe strand comprising a fourth fluorophore and the quencher oligonucleotide;
a second double-stranded probe comprising a second probe strand comprising a fourth fluorophore and a second quencher oligonucleotide;
a second stem-loop probe comprising a fourth fluorophore and the quencher; or
a second stem-loop probe comprising a fourth fluorophore and a second quencher.

In some embodiments, kits comprising a second allele-specific primer also comprise a double-stranded probe comprising a probe strand comprising a second fluorophore and a quencher oligonucleotide (e.g., the probe that is also used with the first allele-specific primer). In some embodiments, kits comprising a second allele-specific primer also comprise a double-stranded probe comprising a probe strand comprising a second fluorophore (e.g., the probe that is also used with the first allele-specific primer).

As described above, in some embodiments, all probes comprise the same donor fluorophore that forms a FRET pair with the fluorophores of each of the acceptor fluorophores of the primers. In some embodiments, different primer-probe pairs use different acceptor-donor FRET pairs. As such, in some embodiments the fluorophore and the second fluorophore are a FRET pair, the third fluorophore and the second fluorophore are a FRET pair, or the third fluorophore and the fourth fluorophore are a FRET pair.

Further, embodiments of methods are provided for detecting a nucleic acid in a sample (e.g., a BRAF nucleic acid), e.g., a method comprising contacting a sample comprising a nucleic acid with a primer (e.g., an allele-specific primer (e.g., an allele-specific primer for a BRAF mutation that encodes a B-Raf protein comprising the amino acid substitution V600E, V600K, and/or V600D), e.g., a primer comprising a fluorophore (e.g., FAM, TET, JOE, HEX, TAMRA, ROX, Cy3, Cy5, and Cy5.5)) in a quenched state (e.g., the primer comprises a double-stranded duplex region and/or the fluorophore is quenched by a quencher (e.g., BHQ-1, BHQ-2, and BHQ-3) when the primer is in the quenched state (e.g., the primer consists of one oligonucleotide comprising a fluorophore and a quencher (e.g., the primer is a stem-loop primer) or the primer consists of a first oligonucleotide comprising a fluorophore and a second oligonucleotide comprising a quencher, wherein the first oligonucleotide is hybridized to the second oligonucleotide (e.g., the primer is a double-stranded linear primer)); a primer (e.g., an allele-specific primer (e.g., an allele-specific primer for a BRAF mutation that encodes a B-Raf protein comprising the amino acid substitution V600E, V600K, and/or V600D)) and a probe (e.g., a probe comprising a fluorophore (e.g., FAM, TET, JOE, HEX, TAMRA, ROX, Cy3, Cy5, and Cy5.5)) in a quenched state (e.g., the probe comprises a double-stranded duplex region and/or the fluorophore is quenched by a quencher (e.g., BHQ-1, BHQ-2, and BHQ-3) when the probe is in the quenched state (e.g., the probe consists of one oligonucleotide comprising a fluorophore and a quencher (e.g., the probe is a stem-loop probe) or the probe consists of a first oligonucleotide comprising a fluorophore and a second oligonucleotide comprising a quencher, wherein the first oligonucleotide is hybridized to the second oligonucleotide (e.g., the probe is a double-stranded linear probe))); or a primer (e.g., an allele-specific primer (e.g., an allele-specific primer for a BRAF mutation that encodes a B-Raf protein comprising the amino acid substitution V600E, V600K, and/or V600D), e.g., a primer comprising a fluorophore (e.g., FAM, TET, JOE, HEX, TAMRA, ROX, Cy3, Cy5, and Cy5.5)) and a probe (e.g., a probe comprising a fluorophore (e.g., FAM, TET, JOE, HEX, TAMRA, ROX, Cy3, Cy5, and Cy5.5)) in an unexcited state; and then detecting a signal (e.g., a fluorescent signal) from the primer in a detectable state if the primer hybridizes to the nucleic acid; detecting a signal (e.g., a fluorescent signal) from the primer in a detectable state if the primer hybridizes to the nucleic acid and the primer is incorporated into an amplicon (e.g., the primer in the quenched state is incorporated into a strand of a nucleic acid by a polymerase); or detecting a signal (e.g., a fluorescent signal) from the primer if the probe hybridizes to a complement of the nucleic acid comprising the primer (e.g., the primer comprises a fluorophore that is a fluorescence resonance energy transfer (FRET) acceptor compatible with the fluorophore of the probe comprising a fluorophore); and, in some embodiments, performing a polymerase chain reaction (e.g., a real-time polymerase chain reaction), wherein the nucleic acid comprises a BRAF gene or a portion of a BRAF gene and the nucleic acid is detected in the sample when the signal is detected.

In some embodiments, the technology provides a method comprising contacting a sample comprising a nucleic acid with a plurality of primers (e.g., a plurality of allele-specific primers (e.g., a plurality of allele-specific primers for one or more BRAF mutations that encode B-Raf proteins comprising an amino acid substitution V600E, V600K, and/or V600D), e.g., a plurality of primers each comprising a fluorophore (e.g., FAM, TET, JOE, HEX, TAMRA, ROX, Cy3, Cy5, and Cy5.5)) in a quenched state (e.g., each primer comprises a double-stranded duplex region and/or the fluorophore of each primer is quenched by a quencher (e.g., BHQ-1, BHQ-2, and BHQ-3) when each primer is in the quenched state (e.g., the primer consists of one oligonucleotide comprising a fluorophore and a quencher (e.g., the primer is a stem-loop primer) or the primer consists of a first oligonucleotide comprising a fluorophore and a second oligonucleotide comprising a quencher, wherein the first oligonucleotide is hybridized to the second oligonucleotide (e.g., the primer is a double-stranded linear primer)); a plurality of primers (e.g., a plurality of allele-specific primers (e.g., a plurality of allele-specific primers for one or more BRAF mutations that encode a B-Raf protein comprising an amino acid substitution V600E, V600K, and/or V600D)) and a probe (e.g., a probe comprising a fluorophore (e.g., FAM, TET, JOE, HEX, TAMRA, ROX, Cy3, Cy5, and Cy5.5)) in a quenched state (e.g., the probe comprises a double-stranded duplex region and/or the fluorophore is quenched by a quencher (e.g., BHQ-1, BHQ-2, and BHQ-3) when the probe is in the quenched state (e.g., the probe consists of one oligonucleotide comprising a fluorophore and a quencher (e.g., the probe is a stem-loop probe) or the probe consists of a first oligonucleotide comprising a fluorophore and a second oligonucleotide comprising a quencher, wherein the first oligonucleotide is hybridized to the second oligonucleotide (e.g., the probe is a double-stranded linear probe))); or a plurality of primers (e.g., a plurality of allele-specific primers (e.g., a plurality of allele-specific primers for one or more BRAF mutations that encode a B-Raf protein comprising the amino acid substitution V600E, V600K, and/or V600D), e.g., each primer comprises a fluorophore (e.g., FAM, TET, JOE, HEX, TAMRA, ROX, Cy3, Cy5, and Cy5.5)) and a probe (e.g., a probe comprising a fluorophore (e.g., FAM, TET, JOE, HEX, TAMRA, ROX, Cy3, Cy5, and Cy5.5)) in an unexcited state; and then detecting one or more signals (e.g., one or more fluorescent signals) from one or more primer of the plurality of primers in a detectable state if one or more primer hybridizes to the nucleic acid; detecting one or more signals (e.g., one or more fluorescent signals) from one or more primer of the plurality of primers in a detectable state if one or more primer hybridizes to the nucleic acid and one or more primer is incorporated into an amplicon (e.g., a primer in the quenched state is incorporated into a strand of a nucleic acid by a polymerase); or detecting one or more signals (e.g., one or more fluorescent signals) from one or more primer if the probe hybridizes to a complement of the nucleic acid comprising a primer of the plurality of primers (e.g., each primer comprises a fluorophore that is a fluorescence resonance energy transfer (FRET) acceptor compatible with the fluorophore of the probe comprising a fluorophore); and, in some embodiments, performing a polymerase chain reaction (e.g., a real-time polymerase chain reaction), wherein the nucleic acid comprises a BRAF gene or a portion of a BRAF gene and the nucleic acid is detected in the sample when the signal is detected.

Embodiments provide kits for detecting a BRAF allele. In some embodiments, a kit comprises a detection reagent for detecting one or more BRAF alleles (e.g., comprising a mutation that encodes a B-Raf protein comprising an amino acid substitution that is V600E, V600K, and/or V600D), e.g., a stem-loop primer comprising a fluorophore and a quencher; a double-stranded linear primer comprising an allele-specific single-stranded primer comprising a fluorophore and a complementary quenching oligonucleotide; a double-stranded linear primer comprising an allele-specific single-stranded primer comprising a fluorophore and a complementary oligonucleotide; an allele-specific primer comprising a fluorophore and a double-stranded probe comprising a probe strand comprising a second fluorophore and a quencher oligonucleotide; an allele-specific primer comprising a fluorophore and a double-stranded probe comprising a probe strand comprising a second fluorophore; an allele-specific primer comprising a fluorophore and a single-stranded probe comprising a second fluorophore; or an allele-specific primer comprising a fluorophore and a stem-loop probe comprising a second fluorophore and a quencher; optionally, a second detection reagent for detecting a second BRAF allele, wherein the second detection reagent comprises a second stem-loop primer comprising a second fluorophore and the quencher or a second quencher, a second double-stranded linear primer comprising a second allele-specific single-stranded primer comprising a second fluorophore and the complementary quenching oligonucleotide or a second complementary quenching oligonucleotide, or a second allele-specific primer comprising a third fluorophore (and, optionally, a second double-stranded probe comprising a second probe strand comprising a fourth fluorophore and the quencher oligonucleotide, a second double-stranded probe comprising a second probe strand comprising a fourth fluorophore and a second quencher oligonucleotide, a second stem-loop probe comprising a fourth fluorophore and the quencher, a second stem-loop probe comprising a fourth fluorophore and a second quencher, and/or a second single-stranded probe comprising a fourth fluorophore); and a control nucleic acid comprising a nucleotide sequence from a BRAF gene or from a portion of a BRAF gene. In some embodiments, kits provided herein comprise two fluorophores that are a FRET pair (e.g., any two (or more) of the fluorophore, second fluorophore, third fluorophore, and fourth fluorophore are a FRET pair).

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a drawing showing an embodiment of the technology.

FIG. 2 is a drawing showing an embodiment of the technology.

FIG. 3 is a drawing showing a schematic for an experiment performed using an embodiment of the technology.

FIG. 4 is a drawing showing a schematic for an experiment performed using an embodiment of the technology.

FIG. 5 is a plot of real-time PCR data collected during testing an embodiment of the technology.

FIG. 6 is a plot of data from real-time PCR experiments performed with an embodiment of the technology to detect BRAF mutant nucleic acids.

FIG. 7 is a plot of data from real-time PCR experiments performed with an embodiment of the technology to detect BRAF mutant nucleic acids.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to nucleic acid detection and particularly, but not exclusively, to methods and compositions for the simultaneous detection of multiple nucleic acids (e.g., multiple alleles such as multiple SNPs). In general, the technology makes use of primers and probes labeled with fluorophores and quenchers as described in more detail below. In some embodiments, the technology provided herein allows the detection, identification, and/or reporting of multiple SNPs or genotypes from one homogeneous amplification reaction.

The section headings used herein are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, “polymorphic sequence” refers to any nucleotide sequence capable of variation and “allele” refers to one such variation. Preferably, such a variation is common in a population of organisms and is inherited in a Mendelian fashion. Such alleles may or may not have associated phenotypes. A “single nucleotide polymorphism” (or “SNP”) is one type of “polymorphic sequence” that is characterized by a sequence variation of only one nucleotide. The term “haplotype” refers to a 5′ to 3′ sequence of nucleotides found at one or more polymorphic sites (preferably, at least two polymorphic sites) in a locus on a single chromosome from an individual. The term “genotype” refers a 5′ to 3′ sequence of nucleotide pair(s) found at one or more polymorphic sites in a locus on a pair of homologous chromosome in an individual. The term “nucleotide variation” refers to a nucleotide polymorphism in a DNA sequence at a particular location among contiguous DNA segments that are otherwise similar in sequence. Such contiguous DNA segments include a gene or any other portion of a chromosome. Examples of nucleotide variation are deletion, insertion, and substitution.

The terms “oligonucleotide” or “polynucleotide” or “nucleotide” or “nucleic acid” refer to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The term “primer” as used herein refers to an oligonucleotide, which is capable of acting as a point of initiation for synthesis when placed under conditions in which synthesis of a primer extension product that is complementary to a nucleic acid strand (template) is induced, e.g., in the presence of nucleotides and an agent for polymerization, such as DNA polymerase, and at a suitable temperature and pH. In some embodiments, the primer is single stranded and in some embodiments the primer is partially or completely double stranded. In some embodiments, the primer is an oligodeoxyribonucleotide.

In some embodiments, the primer exists in several alternative conformations or states, some of which comprise double stranded duplex regions and/or single stranded regions. In some embodiments, the hybridized single-stranded components of a double stranded duplex region dissociate (“melt”) to form a single-stranded primer. In some embodiments, the primer comprises a detectable label (e.g., a fluorophore). In some embodiments the primer can adopt a quenched state (e.g., the fluorophore is quenched, e.g., by a quencher moiety, and the primer is a “quenched primer”) and a detectable (unquenched) state (e.g., the fluorophore is in an unquenched state and the primer is a “detectable primer”); furthermore, in some embodiments, the primer can convert from the quenched state (as used herein, a “quenched primer”) to the detectable state (as used herein, a “detectable primer”) and the primer can convert from the detectable state to the quenched state. For example, in some embodiments the population of primers comprises two states, e.g., the quenched and detectable states that are in equilibrium with one another. A change in the chemical and/or physical environment of the primer may alter the thermodynamics and/or kinetics of the conversion from the quenched state to the detectable state and the conversion from the detectable state to the quenched state such that the population of the primer in the quenched state and the population of the primer in the detectable state are changed.

The primer can comprise naturally occurring dNMP (e.g., dAMP, dGMP, dCMP and dTMP), modified nucleotides, or non-natural nucleotides. The primer can also include ribonucleotides. For example, the primer used in this technology may include nucleotides with backbone modifications such as peptide nucleic acid (PNA) (Egholm et al. (1993) Nature, 365: 566-568), phosphorothioate DNA, phosphorodithioate DNA, phosphoramidate DNA, amide-linked DNA, MMI-linked DNA, 2′-O-methyl RNA, alpha-DNA, and methyl phosphonate DNA, nucleotides with sugar modifications such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and nucleotides having base modifications such as C-5 substituted pyrimidines (substituents including fluoro-, bromo-, chloro-, iodo-, methyl-, ethyl-, vinyl-, formyl-, ethynyl-, propynyl-, alkynyl-, thiazolyl-, imidazolyl-, and pyridyl-), 7-deazapurines with C-7 substituents (substituents including fluoro-, bromo-, chloro-, iodo-, methyl-, ethyl-, vinyl-, formyl-, alkynyl-, alkenyl-, thiazolyl-, imidazolyl-, and pyridyl-), inosine, and diaminopurine.

The primer is sufficiently long to prime the synthesis of extension products in the presence of the agent for polymerization. The exact length of the primers will depend on many factors, including temperature, application, and source of primer. The term “annealing” or “priming” as used herein refers to the apposition of an oligodeoxynucleotide or nucleic acid to a template nucleic acid, whereby the apposition enables the polymerase to polymerize nucleotides into a nucleic acid molecule that is complementary to the template nucleic acid or a portion thereof. The term “hybridizing” as used herein refers to the formation of a double-stranded nucleic acid from complementary single stranded nucleic acids. There is no intended distinction between the terms “annealing” and “hybridizing”, and these terms will be used interchangeably. The sequences of primers may comprise some mismatches, so long as they can be hybridized with templates and serve as primers. The term “substantially complementary” is used herein to signify that the primer is sufficiently complementary to hybridize selectively to a template nucleic acid sequence under the designated annealing conditions or stringent conditions, such that the annealed primer can be extended by a polymerase to form a complementary copy of the template.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification, and isolation of particular gene sequences.

In some embodiments, nucleic acids (e.g., primers and probes) comprise a universal or modified base such as deoxyinosine, inosine, 7-deaza-2′-deoxyinosine, 2-aza-2′-deoxyinosine, 2′-O-Me inosine, 2′-F inosine, deoxy 3-nitropyrrole, 3-nitropyrrole, 2′-O-Me 3-nitropyrrole, 2′-F 3-nitropyrrole, 1-(2′-deoxy-beta-D-ribofuranosyl)-3-nitropyrrole, deoxy 5-nitroindole, 5-nitroindole, 2′-O-Me 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′-O-methoxyethyl nebularine, 2′-O-methoxyethyl 5-nitroindole, 2′-O-methoxyethyl 4-nitro-benzimidazole, 2′-O-methoxyethyl 3-nitropyrrole, and combinations thereof.

The following terms are used to describe the sequence relationships between two or more polynucleotides: “reference sequence”, “sequence identity”, “percentage of sequence identity”, and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

The term “substantially homologous” when used in reference to a double-stranded nucleic acid sequence such as a cDNA or genomic clone refers to any probe that can hybridize to either or both strands of the double-stranded nucleic acid sequence under conditions of low to high stringency as described above.

The term “substantially homologous” when used in reference to a single-stranded nucleic acid sequence refers to any probe that can hybridize (i.e., it is the complement of) the single-stranded nucleic acid sequence under conditions of low to high stringency as described above.

As used herein, the terms “complementary” or “complementarity” are used in reference to polynucleotides (e.g., a sequence of nucleotides) related by the base-pairing rules. For example, the sequence 5′-A-G-T-3′ is complementary to the sequence 3′-T-C-A-5′. Complementarity may be “partial”, in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon hybridization. As used herein, a “complement” of an oligonucleotide, polynucleotide, nucleotide, or nucleic acid refers to an oligonucleotide, polynucleotide, nucleotide, or nucleic acid that is wholly or partially complementary to the oligonucleotide, polynucleotide, nucleotide, or nucleic acid by base-pairing rules.

As used herein, the term “hybridization” or “hybridize” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, the melting temperature (To of the formed hybrid, and the G:C ratio within the nucleic acids. A single molecule that contains pairing of complementary nucleic acids within its structure is said to be “self-hybridized.” An extensive guide to nucleic acid hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays”, Elsevier (1993), which is incorporated by reference.

A “sequence” of a biopolymer (e.g., a nucleic acid) refers to the order and identity of monomer units (e.g., nucleotides, etc.) in the biopolymer. The sequence (e.g., base sequence) of a nucleic acid is typically read in the 5′ to 3′ direction.

The terms “detect”, “detecting”, or “detection” refer to an act of determining the existence or presence of one or more targets (e.g., nucleic acids, amplicons, etc.) in a sample.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is frequently that gene which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, “amplification” of a nucleic acid (DNA, RNA, etc.) refers to increasing the concentration of a particular nucleic acid sequence within a mixture of nucleic acid sequences. An “amplicon” is a target nucleic acid sequence that is amplified. In some embodiments, the amplification is performed in accordance with PCR (polymerase chain reaction). The process for amplifying a DNA molecule by primer annealing, primer extending, and denaturing is well known to those of skill in the art. Suitable annealing or hybridization conditions are routinely determined. Conditions such as temperature, concentration of components, hybridization and washing times, buffer components, and their pH and ionic strength are varied depending on various factors, including the length and GC content of the primer and the target nucleotide sequence. Detailed conditions for hybridization can be found in Joseph Sambrook, et al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999). In some embodiments, a signal is monitored continuously or at multiple discrete time points during the PCR (e.g., a “real-time PCR”) and in some embodiments a signal is monitored after the PCR has completed (sometimes termed an “end-point PCR”). Data for a real-time PCR are often reported as the monitored intensity of a fluorescent reporter moiety as a function of PCR cycle.

Where a mRNA is employed as starting material, a reverse transcription step is useful prior to performing annealing step, details of which are found in Joseph Sambrook, et al., Molecular Cloning, A Laboratory Manual, Cold Spring Harbor Laboratory Press, Cold Spring Harbor, N.Y. (2001); and Noonan, K. F. et al. (1988) Nucleic Acids Res. 16: 10366). For reverse transcription, an oligonucleotide dT primer hybridizable to the poly A tail of mRNA is often used. The oligonucleotide dT primer comprises dTMPs, one or more of which may be replaced with other dNMPs so long as the dT primer can serve as primer. Reverse transcription is performed with a reverse transcriptase that has RNase H activity. If one uses an enzyme having RNase H activity, a separate RNase H digestion step is often omitted by carefully choosing the reaction conditions.

A variety of DNA polymerases can be used in the extension step of the present methods, which includes “Klenow” fragment of E. coli DNA polymerase I, a thermostable DNA polymerase, and a bacteriophage T7 DNA polymerase. Preferably, the polymerase is a thermostable DNA polymerase that is obtained from a variety of bacterial species, including Thermus aquaticus (Taq), Thermus thermophilus (Tth), Thermus filiformis, Thermis flavus (Tfl), Thermococcus literalis, and Pyrococcus furiosus (Pfu). When a polymerization reaction is being conducted, some embodiments provide the components required for such reaction in excess in the reaction vessel. Excess in reference to components of the extension reaction refers to an amount of each component such that the ability to achieve the desired extension is not substantially limited by the concentration of that component. It is desirable to provide to the reaction mixture an amount of required cofactors such as Mg²⁺, dATP, dCTP, dGTP, and dTTP in sufficient quantity to support the degree of the extension desired.

The term “sample” is used in its broadest sense. In one sense it can refer to an animal cell or tissue. In another sense, it is meant to include a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention. Any nucleic acid sample may be used in practicing the present technology, including without limitation eukaryotic, prokaryotic, and viral nucleic acids. In some embodiments, the target nucleic acid represents a sample of genomic DNA isolated from a patient. This DNA may be obtained from any cell source or body fluid. Non-limiting examples of cell sources available in clinical practice include blood cells, buccal cells, cervicovaginal cells, epithelial cells from urine, fetal cells, or any cells present in tissue obtained by biopsy. Body fluids include blood, urine, cerebrospinal fluid, semen, and tissue exudates at the site of infection or inflammation. Nucleic acid is extracted from a cell source or body fluid using any of the numerous methods that are standard in the art. It will be understood that the particular method used to extract nucleic acid will depend on the nature of the source.

As used herein, “multiplex” refers to the simultaneous detection or identification of multiple nucleic acid targets in a single sample.

### Embodiments of the Technology

Although the disclosure herein refers to certain illustrated embodiments, it is to be understood that these embodiments are presented by way of example and not by way of limitation.

### Multiplex Allele-Specific Priming Detection (Masp)

The Masp technology is associated with the following basic concepts (see FIG. 1).

Each allele-specific primer contains a unique fluorophore specific to the primer. In the absence of target, the fluorophore on the primer is quenched by a quencher due to a stem-loop structure or a partially double-stranded structure formed by the free primer (e.g., see Huang (2007) Nucleic Acids Res. 35: e101, incorporated herein by reference in its entirety for all purposes). In the presence of target, the primer binds to the target and is extended, resulting in the separation of the fluorophore from the quencher and the output of a fluorescent signal.

In some approaches, portions of the allele-specific primer are not complementary to the target sequence. For example, an allele-specific primer may have a non-complementary region such as a tail region or a stem-loop primer may be non-complementary to the target, e.g., in the loop region. Or, the double stranded duplex region of a primer may be completely or partially non-complementary to the target sequence in the template or its complementary sequence in the template. In some embodiments associated with these types of primers, the separation of the fluorophore from the quencher is not the result of hybridization, but separation of the fluorophore from the quencher results from extension of the reverse primer on the other side of the target region through the primer. After the allele-specific labeled primer is extended, the reverse primer is extended on the template that was generated from extension of the allele-specific labeled primer. This extension results in the synthesis of a strand complementary to the primer, and, in so doing, the polymerase separates the fluorophore from the quencher as it moves through the double-stranded duplex region (e.g., the duplex is melted). Thus, it is the strand that is extended from the reverse primer that displaces the quenching oligonucleotide in the case of a double-stranded linear primer design or that separates the quencher label from the fluorophore label in the case of a single-stranded stem-loop design due to hybridization of the extended sequence and the loop sequence. Consequently, in some embodiments, separation of the fluorophore and the quencher results from a combination of hybridization and extension.

Design of the stem-loop primers and the partially double-stranded primer structures provides for differentiating the signal between extended and unextended primers and for minimizing or eliminating non-specific priming. The technology is equally applicable to forward primers as it is to reverse primers with the appropriate changes in assay design that are within the ordinary skill of the art. As such, the terms and concepts associated with “primer”, “allele-specific primer”, and/or “forward primer” are applicable to reverse primers. Assays based on this technology are implemented, in some embodiments, in a real-time PCR assay and in some embodiments, in an end-point PCR assay.

### FRET-Mediated Multiplex Allele Specific Priming Detection (F-Masp).

The F-Masp technology is associated with the following basic concepts (FIG. 2). Each allele-specific primer contains a unique FRET acceptor fluorophore specific to the primer. In the absence of the target, no priming occurs and the FRET acceptor does not fluoresce. Also, the FRET donor probe does not bind to the amplicon because it has not been produced due to the absence of amplification. In some embodiments, the donor fluorophore on the probe is quenched by a quencher due to a stem-loop structure or a partially double-stranded structure formed by the free probe (e.g., see Huang (2007) Nucleic Acids Res. 35: e101, incorporated herein by reference in its entirety for all purposes). Alternatively, the probe is a single-stranded probe comprising a donor fluorophore (e.g., it does not comprise a quencher). In such embodiments, the fluorophore is in an unexcited state when no excitation energy is exciting the fluorophore (e.g., the fluorophore is not being excited by a light source, coherent (e.g., laser) source, etc. of the appropriate excitation wavelength).

In the presence of target, the primer hybridizes and is extended, and the probe binds to the extended strand comprising the primer. As a result of the binding, the donor and acceptor are brought together (e.g., within the distance required for FRET coupling), FRET occurs between the donor on the probe and the acceptor on the primer, and the acceptor will fluoresce to produce a signal. The technology is equally applicable to forward primers as it is to reverse primers with the appropriate changes in assay design that are within the ordinary skill of the art. Assays based on this technology are implemented, in some embodiments, in a real-time PCR assay and in some embodiments, in an end-point PCR assay. Also, embodiments also provide for equivalent technologies in which the donor and acceptor of a FRET pair are interchanged, e.g., so that a donor fluorophore is present on a primer and an acceptor fluorophore is present on a probe.

### Allele-Specific PCR Markers

“Allele-specific PCR” is an application of PCR in which alleles that differ by one or more nucleotides can be distinguished on the basis of an amplification product (Ugozzoli and Wallace (1991) Methods: A Companion to Methods in Enzymology 2: 42-48). The technique utilizes primers with specific mismatches at or near the 3′ end that permit preferential amplification of one allele (the target allele) relative to another (the non-target allele) (Ugozzoli and Wallace, supra; Cha et al. (1992) PCR Methods and Applications 2: 14-20). This procedure offers the possibility of generating single nucleotide polymorphism (SNP)-based markers, e.g., for the construction of linkage maps, and represents an excellent option for constructing dense maps composed entirely of these markers. Allele-specific PCR has been used previously in attempts to detect the presence or absence of one or more variant nucleotide sequences by amplification (see, e.g., European Patent Application No. 89302331.7, Publication No. 0332435), including attempts to detect point mutations associated with a variety of genetic diseases (Ugozzoli and Wallace, supra; Wenham et al. (1991) Clinical Chemistry 37: 241-244; Chang (1997) BioTechniques 22: 520-527).

### Stem-Loop Primers

Some embodiments of the technology use an allele-specific stem-loop primer. The stem-loop primer is an oligonucleotide that comprises a fluorophore at or near one of its ends and a quencher at or near the other one of its ends, e.g., the fluorophore is at or near the 5′ end of the oligonucleotide and the quencher is at or near the 3′ end of the oligonucleotide or the quencher is at or near the 5′ end of the oligonucleotide and the quencher is at or near the 3′ end of the oligonucleotide. The quencher or fluorophore at the 3′ end is attached to one of the nucleotides so that the fluorophore is quenched when the stem-loop structure is formed and the primer is not bound to a target and so that the 3′ end provides a priming site (e.g., a 3′ hydroxyl) for the extension of the primer by a polymerase (e.g., in a PCR). The stem-loop primer technology is distinct from stem-loop probes such as molecular beacons in that probes are designed to bind to a target without providing a priming site while the stem-loop primers provided herein bind to a target site and provide a priming site. While some of the concepts are similar, the function of the stem-loop primers in priming the synthesis of a nucleic acid is associated with features of stem-loop primers that are distinct from stem-loop probes, e.g., such as the position of the 3′ moiety (the fluorophore or the quencher).

In some embodiments, the stem-loop primer is complementary to a target sequence comprising a sequence that is desired to be detected, such as a mutation (e.g., a SNP, an insertion, a deletion, a base change, etc.) or a wild-type sequence.

For example, in some embodiments the stem-loop primer is approximately 18 to 50 nucleotides long and comprises three regions: a 5′ stem-forming region of approximately 4 to 10 nucleotides, a central loop region comprising 10 to 30 nucleotides, and a 3′ stem-forming region comprising approximately 4 to 10 nucleotides. The loop region comprises a sequence that is complementary to the target DNA or RNA and does not base pair with itself or other regions of the stem-loop primer; the sequence of the 5′ stem-forming region and the sequence of the 3′ stem-forming region are complementary to each other and form a double stranded “stem” structure when the primer is not bound to a target nucleic acid.

The 3′ stem-forming region and the 5′ stem-forming region have various degrees of complementarity with the target sequence in various embodiments of the technology (e.g., the 3′ stem-forming region and/or the 5′ stem-forming region have no complementarity with the target sequence, the 3′ stem-forming region and/or the 5′ stem-forming region have complete complementarity to the target sequence, the 3′ stem-forming region and/or the 5′ stem-forming region have an intermediate complementarity with the target sequence, e.g., the 3′ stem-forming region and/or the 5′ stem-forming region has 1 or more mismatches or gaps (e.g., insertions or deletions (e.g., that form a bulge) relative to the target sequence, e.g., 1, 2, 3, 4, 5 mismatches or has approximately 10%, 20%, 30%, 40%, 50%, 60%, 70%, 80%, 90% complementarity with the target sequence).

The 3′ stem-forming region and the 5′ stem-forming region are not required to have the same degree of complementarity with the target sequence, but they may have the same degree of complementarity with the target sequence. As such, in some embodiments, 3′ stem-forming region and the 5′ stem-forming region have the same degree of complementarity with the target sequence and in some embodiments the 3′ stem-forming region and the 5′ stem-forming region have different degrees of complementarity with the target sequence. In addition, the 5′ stem-forming region and the 3′ stem-forming region may have any degree of complementarity that is appropriate for the technology (e.g., the sequence of the 5′ stem-forming region and the sequence of the 3′ stem-forming region are completely complementary to each other or comprise 1 or more mismatches or gaps (e.g., insertions or deletions (e.g., that form a bulge) with respect to each other)). The stem duplex may form from a completely complementary 5′ stem-forming region and 3′ stem-forming region or from a 5′ stem-forming region and 3′ stem-forming region having one or more mismatches or gaps.

At one end (e.g., the 5′ end or the 3′ end) of the stem-loop primer, a fluorescent dye is covalently attached. At the other end (e.g., the 3′ end or the 5′ end), a non-fluorescent quencher dye is covalently attached. When the stem-loop primer is in the closed loop conformation, the quencher is near the fluorophore and the quencher quenches (e.g., minimizes, decreases, and/or eliminates) the fluorescence emission of the fluorophore at one or more wavelengths at which fluorescence emission is monitored.

If the nucleic acid to be detected is sufficiently complementary to the sequence of the stem-loop primer to effect hybridization of the nucleic acid to be detected and the stem-loop primer, the stem-loop primer linearizes (e.g., unfolds from the closed loop conformation) and it hybridizes to the target sequence. The duplex formed between the target nucleic acid and the linearized stem-loop primer is more stable than the stem duplex formed by the 3′ stem-forming region and the 5′ stem-forming region of the stem, e.g., in some embodiments because the duplex formed with the target nucleic acid is more thermodynamically stable, e.g., because it involves more base pairs. In this linear conformation, the fluorophore and quencher are separated and the primer is in a state that can be extended by a polymerase under suitable reaction conditions. When the fluorophore is separated from the quencher, the fluorophore fluoresces to produce a signal when queried (e.g., upon absorbing a photon at an excitation wavelength and emitting a photon at one or more monitored emission wavelengths). The emission is associated with hybridization and thus signifies the presence of the target nucleic acid in the test sample. In some embodiments, the fluorescence is detected specifically in the fluorescently-labeled amplicon produced by the method and in some embodiments the general fluorescence of the sample in detected.

When the technology is used in a multiplex assay to detect two or more target nucleic acids (e.g., two or more alleles, SNPs, etc.), two or more allele-specific stem-loop primers are used. The two or more allele-specific stem-loop primers comprise two or more different fluorophores (e.g., that are detectable by different emission spectra and/or by monitoring emission at two wavelengths) and comprise sequences that are specific (e.g., complementary) for the two or more target nucleic acids. The two or more allele-specific stem-loop primers may comprise the same quencher or may comprise different quenchers. The two or more allele-specific stem-loop primers may comprise the same complementary 3′ and 5′ stem-forming regions or may comprise different complementary 3′ and 5′ stem-forming regions. In some embodiments, each allele-specific stem-loop primer is specific for a different target nucleic acid and in some embodiments each allele-specific stem-loop primer is specific for a different group of (e.g., a group comprising two or more) target nucleic acids. As an illustrative and non-limiting example, the four different alleles A, B, C, and D may be detected by four different allele-specific stem-loop primers in some embodiments; or, the alleles A and B may be detected by one allele-specific stem-loop primer and the alleles C and D may be detected by another allele-specific stem-loop primer in some embodiments.

### Double Stranded Linear Primers

Some embodiments of the technology use an allele-specific double-stranded linear primer. The allele-specific double-stranded linear primer comprises two strands: an allele-specific (single-stranded) primer and a complementary quenching oligonucleotide. The allele-specific primer has a length similar to a conventional primer (e.g., 15 to 50 nucleotides) and comprises a covalently-linked fluorophore. The fluorophore is covalently linked to any nucleotide of the allele-specific primer but is typically at or near the 5′ end of the allele-specific primer. The quenching oligonucleotide is typically shorter than the allele-specific primer (e.g., 10 to 20 nucleotides) but may be longer (e.g., 20 nucleotides or more, including lengths as long as or longer than the allele-specific primer). The quenching oligonucleotide comprises a covalently-linked quencher. The quencher is covalently linked to any nucleotide of the quencher oligonucleotide but is typically at or near the 3′ end of the quencher oligonucleotide. When a duplex is formed by the allele-specific primer and the quencher oligonucleotide, the quencher is near the fluorophore and the quencher quenches (e.g., minimizes, decreases, and/or eliminates) the fluorescence emission of the fluorophore at one or more wavelengths at which fluorescence emission is monitored.

The duplex may be of any length up to the length of the shorter of the allele-specific primer and the quencher oligonucleotide. The allele-specific primer and the quencher oligonucleotide may have any degree of complementarity that is appropriate for the technology (e.g., the sequence of the allele-specific primer and the sequence of the quencher oligonucleotide are completely complementary to each other or comprise 1 or more mismatches or gaps (e.g., insertions or deletions (e.g., that form a bulge) with respect to each other)). The duplex may form from a completely complementary allele-specific primer and quencher oligonucleotide or from an allele-specific primer and quencher oligonucleotide having one or more mismatches or gaps.

In some embodiments, the allele-specific primer comprises a tail region that is not complementary to the target and/or is not complementary to the quencher oligonucleotide.

In some embodiments, the quencher oligonucleotide comprises a tail region that is not complementary to the target and/or is not complementary to the allele-specific oligonucleotide. For example, in some embodiments the degree of complementarity between the allele-specific primer and the quencher oligonucleotide and/or the degree of complementarity between the allele-specific primer and the target are designed to control the melting temperatures of the double-stranded duplex region relative and the hybrid formed by the allele-specific primer and target and/or the relative melting temperatures of the double-stranded duplex region relative and the hybrid formed by the allele-specific primer and target.

The allele-specific primer has various degrees of complementarity with the target sequence in various embodiments of the technology (e.g., the allele-specific primer has complete complementarity to the target sequence or has 1 or more mismatches or gaps (e.g., insertions or deletions (e.g., that form a bulge) relative the target sequence). In some embodiments for the specific detection of a target nucleic acid (e.g., an allele such as a SNP, the allele-specific primer is completely complementary to the target nucleic acid).

A fluorescent dye is covalently attached to the allele-specific primer. A non-fluorescent quencher dye is covalently attached to the quencher oligonucleotide. When the allele-specific primer and the quencher oligonucleotide form a duplex (e.g., in the absence of the target), the quencher is near the fluorophore and the quencher quenches (e.g., minimizes, decreases, and/or eliminates) the fluorescence emission of the fluorophore at one or more wavelengths at which fluorescence emission is monitored.

If the nucleic acid to be detected (e.g., the target nucleic acid) is sufficiently complementary to the sequence of the allele-specific primer to effect hybridization of the nucleic acid to be detected and the allele-specific primer, the allele-specific primer separates from the quencher oligonucleotide (e.g., the duplex formed from the allele-specific primer and the quencher oligonucleotide melts) and the allele-specific primer hybridizes to the target sequence. The duplex formed between the target nucleic acid and the allele-specific primer is more stable than the duplex formed from the allele-specific primer and the quencher oligonucleotide, e.g., in some embodiments because the duplex formed with the target nucleic acid is more thermodynamically stable, e.g., because it involves more base pairs. In this conformation, the fluorophore and quencher are separated and the allele-specific primer is in a state that can be extended by a polymerase under suitable reaction conditions.

In some approaches, portions of the allele-specific primer are not complementary to the target sequence. For example, an allele-specific primer may comprise a non-complementary region such as a tail region. Or, the double stranded duplex region of a primer may be completely or partially non-complementary to the target sequence in the template or its complementary sequence in the template. In some embodiments associated with these types of primers, the separation of the fluorophore from the quencher is not the result of hybridization, but separation of the fluorophore from the quencher results from extension of the reverse primer on the other side of the target region through the incorporated primer. After the allele-specific labeled primer is extended, the reverse primer is extended on the template that was generated from extension of the allele-specific labeled primer. This extension results in the synthesis of a strand complementary to the primer, and, in so doing, the polymerase separates the fluorophore from the quencher as it moves through the double-stranded duplex region (e.g., the duplex is melted). Thus, it is the strand that is extended from the reverse primer that displaces the quenching oligonucleotide of a double-stranded linear primer. Consequently, in some embodiments, separation of the fluorophore and the quencher results from a combination of hybridization and extension.

When the fluorophore is separated from the quencher, the fluorophore will fluoresce to produce a signal when queried (e.g., upon absorbing a photon at an excitation wavelength and emitting a photon at one or more monitored emission wavelengths). The emission is associated with hybridization and thus signifies the presence of the target nucleic acid in the test sample. In some embodiments, the fluorescence is detected specifically in the fluorescently-labeled amplicon produced by the method and in some embodiments the general fluorescence of the sample in detected.

When the technology is used in a multiplex assay to detect two or more target nucleic acids (e.g., two or more alleles, SNPs, etc.), two or more allele-specific double-stranded linear primers are used. The two or more allele-specific double-stranded linear primers comprise two or more different fluorophores (e.g., that are detectable by different emission spectra and/or by monitoring emission at two wavelengths) and comprise sequences that are specific (e.g., complementary) for the two or more target nucleic acids. The two or more allele-specific double-stranded linear primers may comprise the same quencher or may comprise different quenchers. The two or more allele-specific double-stranded linear primers may comprise the same complementary duplex-forming regions or may comprise different complementary duplex-forming regions. In some embodiments, each allele-specific double-stranded linear primer is specific for a different target nucleic acid and in some embodiments each allele-specific double-stranded linear primer is specific for a different group of (e.g., a group comprising two or more) target nucleic acids. As an illustrative and non-limiting example, the four different alleles A, B, C, and D may be detected by four different allele-specific double-stranded linear primers in some embodiments; or, the alleles A and B may be detected by one allele-specific double-stranded linear primer and the alleles C and D may be detected by another allele-specific double-stranded linear primer in some embodiments.

### F-Masp

In some embodiments, e.g., a FRET-mediated multiplex allele-specific priming detection (F-Masp), an allele-specific primer and a double-stranded probe are used. In some embodiments, an allele-specific primer and a single-stranded probe are used. The allele-specific primer comprises a fluorescent label (e.g., an acceptor fluorophore). The label is covalently bound to the allele-specific primer. The label is linked to any nucleotide of the allele-specific primer and is typically near the 3′ end of the allele-specific primer.

The allele-specific primer has a length similar to a conventional primer (e.g., 15 to 50 nucleotides) and comprises a covalently-linked fluorophore. The fluorophore is covalently linked to any nucleotide of the allele-specific primer but is typically at or near the 3′ end of the allele-specific primer. The allele-specific primer has various degrees of complementarity with the target sequence in various embodiments of the technology (e.g., the allele-specific primer has complete complementarity to the target sequence or has 1 or more mismatches or gaps (e.g., insertions or deletions (e.g., that form a bulge) relative the target sequence). In some embodiments for the specific detection of a target nucleic acid (e.g., an allele such as a SNP), the allele-specific primer is completely complementary to the target nucleic acid.

The single-stranded probe comprises a probe strand. The probe strand has a length similar to a conventional probe (e.g., 15 to 100 or more nucleotides) and comprises a covalently-linked fluorophore (e.g., a donor fluorophore). The fluorophore is covalently linked to any nucleotide of the probe strand but is typically at or near the 3′ end of the probe strand. In such embodiments, the fluorophore and/or the probe are in an “unexcited” state when no excitation energy is exciting the fluorophore (e.g., the fluorophore is not being excited by a light source, coherent (e.g., laser) source, etc. of the appropriate excitation wavelength). In this state, the fluorophore also does not transfer energy to an acceptor of a FRET pair and thus no signal is detected.

The double-stranded probe comprises two strands: a probe strand and a complementary quenching oligonucleotide (see, e.g., Huang (2007) Nucleic Acids Res. 35: e101, incorporated herein by reference in its entirety for all purposes). The probe strand has a length similar to a conventional probe (e.g., 15 to 100 or more nucleotides) and comprises a covalently-linked fluorophore (e.g., a donor fluorophore). The fluorophore is covalently linked to any nucleotide of the probe strand but is typically at or near the 3′ end of the probe strand. The quenching oligonucleotide is typically shorter than the probe strand (e.g., 10 to 20 nucleotides) but may be longer (e.g., 20 nucleotides or more, including lengths as long as or longer than the probe strand). The quenching oligonucleotide comprises a covalently-linked quencher. The quencher is covalently linked to any nucleotide of the quencher oligonucleotide but is typically at or near the 3′ end of the quencher oligonucleotide. When a duplex is formed by the probe strand and the quencher oligonucleotide, the quencher is near the fluorophore and the quencher quenches (e.g., minimizes, decreases, and/or eliminates) the fluorescence emission of the fluorophore at one or more wavelengths at which the fluorophore emits radiation.

The duplex may be of any length up to the length of the shorter of the probe strand and the quencher oligonucleotide. The probe strand and the quencher oligonucleotide may have any degree of complementarity that is appropriate for the technology (e.g., the sequence of the probe strand and the sequence of the quencher oligonucleotide are completely complementary to each other or comprise 1 or more mismatches or gaps (e.g., insertions or deletions (e.g., that form a bulge) with respect to each other)). The duplex may form from a completely complementary probe strand and quencher oligonucleotide or from a probe strand and quencher oligonucleotide having one or more mismatches or gaps.

The probe strand has various degrees of complementarity with the target sequence in various embodiments of the technology (e.g., the probe strand has complete complementarity to the target sequence or has 1 or more mismatches or gaps (e.g., insertions or deletions (e.g., that form a bulge) relative the target sequence). It is preferred that the probe strand is completely complementary to the target nucleic acid over a sequence adjacent to or near to the sequence to which binds the allele-specific primer.

For embodiments of probes comprising a quencher oligonucleotide, if the nucleic acid to be detected (e.g., the target nucleic acid) is sufficiently complementary to the sequence of the probe strand to effect hybridization of the probe strand and the target nucleic acid, the probe strand separates from the quencher oligonucleotide (e.g., the duplex formed from the probe strand and the quencher oligonucleotide melts) and the probe strand hybridizes to the target sequence. The duplex formed between the target nucleic acid and the probe strand is more stable than the duplex formed from the probe strand and the quencher oligonucleotide, e.g., in some embodiments because the duplex formed with the target nucleic acid is more thermodynamically stable, e.g., because it involves more base pairs. In this conformation, the fluorophore and quencher are separated and the fluorophore can emit radiation, e.g., to excite the acceptor fluorophore. In some embodiments, the probe is designed to be non-extendable, e.g., the 3′ end (3′ hydroxyl) is blocked or otherwise prevented from being extended by a polymerase (e.g., by the addition of a nucleotide to the probe 3′ end).

For embodiments of probes that are single-stranded (e.g., that do not comprise a quencher nucleotide), if the nucleic acid to be detected (e.g., the target nucleic acid) is sufficiently complementary to the sequence of the probe strand to effect hybridization of the probe strand and the target nucleic acid, the probe strand hybridizes to the target sequence. In this conformation, and the fluorophore can excite the acceptor fluorophore. In some embodiments, the probe is designed to be non-extendable, e.g., the 3′ end (3′ hydroxyl) is blocked or otherwise prevented from being extended by a polymerase (e.g., by the addition of a nucleotide to the probe 3′ end).

The fluorescent label of the allele-specific primer and the fluorescent label of the probe strand form a FRET pair. A FRET pair comprises two fluorophores having emission and excitation characteristics whereby one fluorophore is a donor fluorophore and the other fluorophore is an acceptor fluorophore. The donor fluorophore, initially in its electronic excited state, transfers energy to the acceptor fluorophore through nonradiative dipole-dipole coupling. The emission spectrum of the donor fluorophore overlaps to some extent with the excitation spectrum of the acceptor fluorophore. The efficiency of transfer depends on the distance between the donor and the acceptor, the spectral overlap of the donor emission spectrum and the acceptor absorption spectrum, and the relative orientation of the donor emission dipole moment and the acceptor absorption dipole moment. In particular, the efficiency of the energy transfer is inversely proportional to the sixth power of the distance between the donor and acceptor. As such, a detectable energy transfer occurs when the acceptor and donor are separated by a very small distance (e.g., a distance of approximately 1 to 10 nm).

FRET occurs when 1) the allele-specific primer is incorporated into a strand of an amplicon by a polymerase, and 2) the probe strand binds to the strand of the amplicon comprising the fluorophore. Because the amplicon is produced from an allele-specific primer comprising a fluorophore, binding of the probe strand to the amplicon places the fluorophore of the allele-specific primer within the appropriate distance of the fluorophore of the probe strand for energy transfer to occur. As such, the emission of the acceptor fluorophore is detectable (e.g., by monitoring emission at one or more wavelengths) and signals the presence of the target nucleic acid in the sample. In some embodiments, the F-Masp technology comprising the detection of a signal upon incorporation of the allele-specific primer and the binding of the probe strand provides a more-sensitive and/or more specific technology than the Masp embodiments.

When the technology is used in a multiplex assay to detect two or more target nucleic acids (e.g., two or more alleles, SNPs, etc.), two or more allele-specific primers are used. The two or more allele-specific primers comprise two or more different fluorophores (e.g., acceptor fluorophores that are detectable by different emission spectra and/or by monitoring emission at two wavelengths) and comprise sequences that are specific (e.g., complementary) for the two or more target nucleic acids. In some embodiments, the same probe strand is used to detect the two or more target nucleic acids and in some embodiments, a different probe strand is used to detect the two or more target nucleic acids. The composition (e.g., length, sequence, etc.) of the probe strand depends on the sequence of the target nucleic acid adjacent to or near the target site where the allele-specific primer binds. The fluorophores (e.g., the donor fluorophores) of the probe strands may be the same or may be different. The fluorophores (e.g., the donor fluorophores) of the probe strands act as donors for the two different fluorophores attached to the two or more allele-specific primers. In some embodiments, each allele-specific primer is specific for a different target nucleic acid and in some embodiments each allele-specific primer is specific for a different group of (e.g., a group comprising two or more) target nucleic acids. As an illustrative and non-limiting example, the four different alleles A, B, C, and D may be detected by four different allele-specific primers in some embodiments; or, the alleles A and B may be detected by one allele-specific primer and the alleles C and D may be detected by another allele-specific primer in some embodiments.

### Molecular Beacon F-Masp

In some embodiments of the F-Masp technology, a molecular beacon probe is used instead of the double-stranded probe (see, e.g., Tyagi et al. (1996) Nat. Biotechnol. 14: 303; Drake et al. (2004) Appl. Spectrosc. 58: 269A, incorporated herein by reference in their entireties for all purposes). As such, the technology includes embodiments in which the term “double-stranded probe” is replaced with the term “molecular beacon probe” in the F-Masp technology described herein and as understood by one of ordinary skill in the art.

The molecular beacon probe is an oligonucleotide that comprises a fluorophore at or near one of its ends and a quencher at or near the other one of its ends, e.g., the fluorophore is at or near the 5′ end of the oligonucleotide and the quencher is at or near the 3′ end of the oligonucleotide or the quencher is at or near the 5′ end of the oligonucleotide and the quencher is at or near the 3′ end of the oligonucleotide. The quencher or fluorophore at the 3′ end is attached to one of the nucleotides so that the fluorophore is quenched when the stem-loop structure is formed and the probe is not bound to a target.

For example, in some embodiments the molecular beacon probe is approximately 18 to 50 nucleotides long and comprises three regions: a 5′ stem-forming region of approximately 4 to 10 nucleotides, a central loop region comprising 10 to 30 nucleotides, and a 3′ stem-forming region comprising approximately 4 to 10 nucleotides. The loop region comprises a sequence that is complementary to the target DNA or RNA and does not base pair with itself or other regions of the molecular beacon probe; the sequence of the 5′ stem-forming region and the sequence of the 3′ stem-forming region are sufficiently complementary to each other to form a double stranded “stem” structure when the molecular beacon probe is not bound to a target nucleic acid.

The 3′ stem-forming region and the 5′ stem-forming region have various degrees of complementarity with the target sequence in various embodiments of the technology (e.g., the 3′ stem-forming region and/or the 5′ stem-forming region have no complementarity with the target sequence, the 3′ stem-forming region and/or the 5′ stem-forming region have complete complementarity to the target sequence, the 3′ stem-forming region and/or the 5′ stem-forming region have an intermediate complementarity with the target sequence, e.g., the 3′ stem-forming region and/or the 5′ stem-forming region has 1 or more mismatches or gaps (e.g., insertions or deletions (e.g., that form a bulge) relative to the target sequence, e.g., 1, 2, 3, 4, 5 mismatches or has approximately 10%, 20%, 30%, 40%, 50%, 60%, 70%, 80%, 90% complementarity with the target sequence).

The 3′ stem-forming region and the 5′ stem-forming region are not required to have the same degree of complementarity with the target sequence, but they may have the same degree of complementarity with the target sequence. As such, in some embodiments, the 3′ stem-forming region and the 5′ stem-forming region have the same degree of complementarity with the target sequence and in some embodiments the 3′ stem-forming region and the 5′ stem-forming region have different degrees of complementarity with the target sequence. In addition, the 5′ stem-forming region and the 3′ stem-forming region may have any degree of complementarity that is appropriate for the technology (e.g., the sequence of the 5′ stem-forming region and the sequence of the 3′ stem-forming region are completely complementary to each other or comprise 1 or more mismatches or gaps (e.g., insertions or deletions (e.g., that form a bulge) with respect to each other)). The stem duplex may form from a completely complementary 5′ stem-forming region and 3′ stem-forming region or from a 5′ stem-forming region and 3′ stem-forming region having one or more mismatches or gaps.

At one end (e.g., the 5′ end or the 3′ end) of the molecular beacon probe, a fluorescent dye is covalently attached. At the other end (e.g., the 3′ end or the 5′ end), a non-fluorescent quencher dye is covalently attached. When the molecular beacon probe is in the closed loop conformation, the quencher is near the fluorophore and the quencher quenches (e.g., minimizes, decreases, and/or eliminates) the fluorescence emission of the fluorophore (e.g., the donor fluorophore).

If the nucleic acid to be detected is sufficiently complementary to the sequence of the molecular beacon probe to effect hybridization of the nucleic acid to be detected and the molecular beacon probe, the molecular beacon probe linearizes (e.g., unfolds from the closed loop conformation) and it hybridizes to the target sequence. The duplex formed between the target nucleic acid and the linearized molecular beacon probe is more stable than the stem duplex formed by the 3′ stem-forming region and the 5′ stem-forming region of the stem, e.g., in some embodiments because the duplex formed with the target nucleic acid is more thermodynamically stable, e.g., because it involves more base pairs. In this linear conformation, the fluorophore and quencher are separated and the molecular beacon probe is in a state that excites the acceptor fluorophore linked to the allele-specific primer when the two fluorophores are within an appropriate distance for energy transfer (e.g., approximately 1 to 10 nm).

The molecular beacon probe embodiments find use in the multiplex detection of multiple target nucleic acids analogously to the embodiments described herein that use the double stranded probes. When the technology is used in a multiplex assay to detect two or more target nucleic acids (e.g., two or more alleles, SNPs, etc.), two or more allele-specific primers are used. The two or more allele-specific primers comprise two or more different fluorophores (e.g., acceptor fluorophores that are detectable by different emission spectra and/or by monitoring emission at two wavelengths) and comprise sequences that are specific (e.g., complementary) for the two or more target nucleic acids. In some embodiments, the same molecular beacon probe is used to detect the two or more target nucleic acids and in some embodiments, a different molecular beacon probe is used to detect the two or more target nucleic acids. The composition (e.g., length, sequence, etc.) of the molecular beacon probe depends on the sequence of the target nucleic acid adjacent to or near the target site where the allele-specific primer binds. The fluorophores (e.g., the donor fluorophores) of the molecular beacon probe may be the same or may be different. The fluorophores (e.g., the donor fluorophores) of the molecular beacon probe act as donors for the two different fluorophores attached to the two or more allele-specific primers. In some embodiments, each allele-specific primer is specific for a different target nucleic acid and in some embodiments each allele-specific primer is specific for a different group of (e.g., a group comprising two or more) target nucleic acids. As an illustrative and non-limiting example, the four different alleles A, B, C, and D may be detected by four different allele-specific primers in some embodiments; or, the alleles A and B may be detected by one allele-specific primer and the alleles C and D may be detected by another allele-specific primer in some embodiments.

### Fluorescent Moieties

In some embodiments, the oligonucleotides (e.g., stem-loop primers, allele-specific primers, double-stranded linear primers, and/or molecular beacons) used in the technologies described comprise a fluorescent moiety (e.g., an organic dye). A wide variety of fluorescent moieties is known in the art. Examples of compounds that may be used as the fluorescent moiety include but are not limited to xanthene, anthracene, cyanine, porphyrin, and coumarin dyes. Examples of xanthene dyes that find use with the present technology include but are not limited to fluorescein, 6-carboxyfluorescein (6-FAM), 5-carboxyfluorescein (5-FAM), 5- or 6-carboxy-4,7,2′,7′-tetrachlorofluorescein (TET), 5- or 6-carboxy-4′5′2′4′5′7′ hexachlorofluorescein (HEX), 5′ or 6′-carboxy-4′,5′-dichloro-2,′7′-dimethoxyfluorescein (JOE), 5-carboxy-2′,4′,5′,7′-tetrachlorofluorescein (ZOE), rhodol, rhodamine, tetramethylrhodamine (TAMRA), 4,7-dichlorotetramethyl rhodamine (DTAMRA), rhodamine X (ROX), and Texas Red. Examples of cyanine dyes that may find use with the present invention include but are not limited to Cy 3, Cy 3.5, Cy 5, Cy 5.5, Cy 7, and Cy 7.5. Other fluorescent moieties and/or dyes that find use with the present technology include but are not limited to energy transfer dyes, composite dyes, and other aromatic compounds that give fluorescent signals. In some embodiments, the fluorescent moiety comprises a quantum dot.

As such, according to the technology, exemplary fluorophores and dyes that find use include, without limitation, fluorescent dyes or molecules that quench the fluorescence of the fluorescent dyes. Fluorescent dyes include, without limitation, d-Rhodamine acceptor dyes including Cy5, dichloro[R110], dichloro[R6G], dichloro[TAMRA], dichloro[ROX] or the like, fluorescein donor dyes including fluorescein, 6-FAM, 5-FAM, or the like; Acridine including Acridine orange, Acridine yellow, Proflavin, pH 7, or the like; Aromatic Hydrocarbons including 2-Methylbenzoxazole, Ethyl p-dimethylaminobenzoate, Phenol, Pyrrole, benzene, toluene, or the like; Arylmethine Dyes including Auramine O, Crystal violet, Crystal violet, glycerol, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dyes including 1,1′-diethyl-2,2′-cyanine iodide, Cryptocyanine, Indocarbocyanine (C3) dye, Indodicarbocyanine (C5) dye, Indotricarbocyanine (C7) dye, Oxacarbocyanine (C3) dye, Oxadicarbocyanine (C5) dye, Oxatricarbocyanine (C7) dye, Pinacyanol iodide, Stains all, Thiacarbocyanine (C3) dye, ethanol, Thiacarbocyanine (C3) dye, n-propanol, Thiadicarbocyanine (C5) dye, Thiatricarbocyanine (C7) dye, or the like; Dipyrrin dyes including N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminostyryl)-4H-pyran (DCM), acetonitrile, 4-(dicyanomethylene)-2-methyl-6-(p-dimethylaminostyryl)-4H-pyran (DCM), methanol, 4-Dimethylamino-4′-nitrostilbene, Merocyanine 540, or the like; Miscellaneous Dyes including 4′,6-Diamidino-2-phenylindole (DAPI), dimethylsulfoxide, 7-Benzylamino-4-nitrobenz-2-oxa-1,3-diazole, Dansyl glycine, Dansyl glycine, dioxane, Hoechst 33258, DMF, Hoechst 33258, Lucifer yellow CH, Piroxicam, Quinine sulfate, Quinine sulfate, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, methanol, Nile Red, ethanol, Oxazine 1, Oxazine 170, or the like; Polycyclic Aromatic Hydrocarbons including 9,10-Bis(phenylethynyl)anthracene, 9,10-Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1,6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridypruthenium(II), Tetrapyrrole, Bilirubin, Chlorophyll a, diethyl ether, Chlorophyll a, methanol, Chlorophyll b, Diprotonated-tetraphenylporphyrin, Hematin, Magnesium octaethylporphyrin, Magnesium octaethylporphyrin (MgOEP), Magnesium phthalocyanine (MgPc), PrOH, Magnesium phthalocyanine (MgPc), pyridine, Magnesium tetramesitylporphyrin (MgTMP), Magnesium tetraphenylporphyrin (MgTPP), Octaethylporphyrin, Phthalocyanine (Pc), Porphin, ROX, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), pyridine, Zinc tetramesitylporphyrin (ZnTMP), Zinc tetramesitylporphyrin radical cation, Zinc tetraphenylporphyrin (ZnTPP), or the like; Xanthenes including Eosin Y, Fluorescein, basic ethanol, Fluorescein, ethanol, Rhodamine 123, Rhodamine 6G, Rhodamine B, Rose bengal, Sulforhodamine 101, or the like; or mixtures or combination thereof or synthetic derivatives thereof.

Fluorescence resonance energy transfer (FRET) is a distance-dependent interaction between the electronic excited states of two dye molecules in which excitation is transferred from a donor molecule to an acceptor molecule without emission of a photon. In some embodiments, the oligonucleotides (e.g., stem-loop primers, allele-specific primers, double-stranded linear primers, and/or molecular beacons) used in the technologies described comprise a moiety that is appropriate for use in FRET (e.g., a member of a FRET pair, e.g., a FRET donor or a FRET acceptor). Some suitable FRET pairs are provided in Table 1:

TABLE 1
FRET pairs

| Donor | Acceptor |
|---|---|
| FITC | TRITC |
| Cy3 | Cy5 |
| fluorescein | tetramethylrhodamine |
| IAEDANS | fluorescein |
| EDANS | dabcyl |
| fluorescein | fluorescein |
| BODIPY FL | BODPY FL |
| fluorescein | QSY7 |
| fluorescein | QSY9 |

### Quenchers

In some embodiments, the oligonucleotides (e.g., stem-loop primers, allele-specific primers, double-stranded linear primers, and/or molecular beacons) used in the technologies described comprise a quencher moiety. A wide variety of quencher moieties is known in the art. For example, in some embodiments an oligonucleotide comprises a quencher than is a Black Hole Quencher (e.g., BHQ-0, BHQ-1, BHQ-2, BHQ-3), a Dabcy1, an Iowa Black Quencher (e.g., Iowa Black FQ, Iowa Black RQ), an Eclipse quencher.

In some embodiments a BHQ-1 is used with a fluorescent moiety that has an emission wavelength from approximately 500-600 nm. In some embodiments a BHQ-2 is used with a fluorescent moiety that has an emission wavelength from approximately 550-675 nm. In some embodiments, a FRET pair is a fluorophore-quencher pair that provides quenching.

Some exemplary fluorophore-quencher pairs include FAM and BHQ-1, TET and BHQ-1, JOE and BHQ-1, HEX and BHQ-1, Cy3 and BHQ-2, TAMRA and BHQ-2, ROX and BHQ-2, Cy5 and BHQ-3, Cy5.5 and BHQ-3, FAM and BHQ-1, TET and BHQ-1, JOE and 3′-BHQ-1, HEX and BHQ-1, Cy3 and BHQ-2, TAMRA and BHQ-2, ROX and BHQ-2, Cy5 and BHQ-3, Cy5.5 and BHQ-3, or similar fluorophore-quencher pairs available from other commercial entities such as Biosearch Technologies, Inc. of Novato, Calif.

### Kits

Also provided herein are kits comprising one or more compositions described herein. In some embodiments, the kits find use in performing one or more of the methods provided herein. In some embodiments, methods are described in a set of instructions for using the methods and compositions described herein and provided in the kit. Compositions are provided in one or more containers (e.g., bottles, vials, ampules, tubes, etc.) and may be in a ready-to-use format or may be in a format that may be reconstituted (e.g., in a lyophilized format to which water is added. The water may be provided by the kit or by the user.) The kits may include one or more optical filters for use with fluorophores described herein. In some embodiments, the kits comprise a control. Examples of controls are positive controls, e.g., a nucleic acid comprising the one or more target sequence(s) to which the compositions and/or methods of the kit are directed for use, and negative controls, e.g., a nucleic acid that does not comprise the one or more target sequence(s) to which the compositions and/or methods of the kit are directed for use. In some embodiments, controls are provided that are nucleic acids comprising a sequence that is a wild-type allele and/or nucleic acids comprising one or more sequences that are mutated relative to the wild-type sequence (e.g., comprising one or more mutant alleles, SNPs, etc.) In embodiments related to PCR or real-time PCR, kits may comprise a polymerase, a buffer, one or more nucleotides (dNTPs, e.g., dATP, dCTP, dGTP, dTTp, and/or analogues or derivatives thereof), and other reagents. Some kit embodiments provide compositions according to the technology that are pre-dispensed and ready to use in a multiplex format, e.g., in a multiwell plate such as a 96-well plate, a 384-well plate, a 1536-well plate, or a plate comprising more or fewer wells according to the number of tests to be run. In some embodiments, a sample is added to the ready-to-use plate and the user performs an assay, e.g., a PCR or real-time PCR, by thermocycling the plate.

The embodiments of the technology are further understood and described below in the related examples.

### EXAMPLES

### Example 1

### Multiplex Detection of BRAF Mutations

During the developments of embodiments of the technology, data were collected in assays to detect BRAF mutations. BRAF is a human gene that makes a protein called B-Raf. The gene is a proto-oncogene in the serine/threonine-protein kinase family and has been shown to comprise amino acid substitutions in human cancers such as non-Hodgkin lymphoma, colorectal cancer, malignant melanoma, papillary thyroid carcinoma, non-small-cell lung carcinoma, and adenocarcinoma of the lung. In addition, certain other substitutions in B-Raf cause birth defects.

More than 30 mutations of the BRAF gene associated with human cancers have been identified. In 90% of the cases, thymine is substituted with adenine at nucleotide 1799 in exon 15 of the BRAF gene. This leads to valine (V) being substituted for by glutamate (E) at codon 600 (now referred to as V600E) in the activation segment that has been found in human cancers. This mutation has been widely observed in papillary thyroid carcinoma, colorectal cancer, melanoma and non-small-cell lung cancer. Other mutations in BRAF cause substitutions of lysine (K) and aspartic acid (D) for the valine at codon 600, denoted as the V600K and V600D mutations, respectively.

To detect nucleic acids associated with these cancer-causing B-Raf proteins, allele-specific primers were designed according to the technology to detect the mutations in BRAF that result in the V600E, V600K, and V600D substitutions in the B-Raf protein. A real-time PCR used the three allele-specific forward primers (AS-FP) with one common reverse primer (RP). The three allele-specific primers were each labeled with a different fluorophore and one PCR was used to detect the three mutant forms of BRAF in a multiplexed assay. Thus, the SNP mutations are differentiated by monitoring fluorescence emission in three different fluorescent channels. Primers directed to amplify a portion of the BRAF gene exon 13 were designed as an internal endogenous control. See, e.g., FIG. 3.

In particular, the three allele-specific primers were double-stranded primers as described herein. The double stranded primers consisted of a primer strand comprising a fluorophore and a quencher strand comprising a quencher. When the primer comprises the double-stranded duplex region (e.g., in the not-hybridized state and in the incorporated state before the duplex is melted), the primer fluorophore is quenched and the primer is not detectable. See, e.g., FIG. 4, in which the star-shaped element is a fluorophore and the “Q” is a quencher.

Test samples were mixed to have a known amount of BRAF mutant nucleic acid. In particular, samples comprised wild-type BRAF nucleic acid and a mutant nucleic acid at concentrations including 0.5%, 1%, 5%, 25%, and 50%. Real-time PCR was performed with the allele-specific primers to detect the mutant nucleic acid at these levels in the background of wild-type nucleic acid.

One set of experiments collected data to verify the specificity and sensitivity of the double-stranded allele-specific primers. The results of one non-multiplexed experiment to verify the specificity and sensitivity of a double-stranded allele-specific primer to detect V600E are shown in FIG. 5. The fluorescence signal is plotted as a function of cycle number. As shown by the plot, detecting mutant nucleic acid by the mutant allele-specific primer occurs at an earlier cycle than wild-type BRAF is detected by the mutant allele-specific primer. The cycle number of detection depends on the amount of mutant nucleic acid present in the sample. As such, a sample consisting of 50% V600E BRAF is detected at an earlier cycle than a sample consisting of 0.5% BRAF, and both are detected at an earlier cycle than wild-type BRAF is detected. The inset shows the signal from the positive internal control PCR.

A second set of experiments was directed at collecting data from a multiplexed detection of V600E and V600K in cellular DNA. A sample consisting of 10 ng total input cellular DNA was assayed by an embodiment of the technology provided herein. Reaction mixtures comprised a V600E allele-specific primer, a V600K allele-specific primer, wild-type BRAF DNA, and one of % V600E mutant DNA at 0.5%; V600E DNA at 50%; V600K DNA at 1%; or V600K DNA at 50%. Real-time PCR data were collected and plotted in FIG. 6. In FIG. 6, the Y-axis shows the difference in the fluorescence signal between the test sample and the internal endogenous control (dCt) as a function of cycle number. As shown by FIG. 6, both V600E mutant DNA and V600K mutant DNA are detected when present at 1% or less in the sample. In particular, the data show a statistically significant difference in the cycle when the mutants are detected relative to wild-type. The V600E mutant is detected at a dCt of 13.30 when present at 0.5% and at a dCt of 5.35 when present at 50%, while the wild-type was detected at a dCt of 18.80. Likewise, the V600K mutant is detected at a dCt of 9.33 when present at 1% and at a dCt of 3.85 when present at 50%, while the wild-type was detected at a dCt of 20.77.

A similar experiment was performed using 10 ng of total DNA extracted from synthetic formalin-fixed paraffin-embedded (FFPE) tissue samples. FIG. 7 shows the data from this experiment. The data show the detection of V600E mutant nucleic acid extracted from FFPE samples when present at 0.5% in the sample. V600E mutant nucleic acid was detected at a dCt of 10.06 and wild-type BRAF nucleic acid was detected at a dCt of 11.59.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method for detecting a nucleic acid in a sample, the method comprising:
1) contacting a sample comprising a nucleic acid with:
a) a primer in a quenched state;
b) a primer and a probe in a quenched state; or
c) a primer and a probe in an unexcited state; and
2) detecting:
a) a signal from the primer in a detectable state if:
i) the primer hybridizes to the nucleic acid; or
ii) the primer hybridizes to the nucleic acid and the primer is incorporated into an amplicon; or
b) a signal from the primer if the probe hybridizes to a complement of the nucleic acid comprising the primer,
wherein the nucleic acid comprises a BRAF gene or a portion of a BRAF gene and the nucleic acid is detected in the sample when the signal is detected.

2. The method of claim 1 wherein the primer in the quenched state or the probe in the quenched state comprises a double-stranded duplex region.

3. The method of claim 1 wherein the signal is fluorescence.

4. The method of claim 1 wherein:
a) a primer or a probe comprises a fluorophore; or
b) a primer or a probe comprises a fluorophore and the fluorophore is quenched by a quencher when the primer or the probe is in the quenched state.

5. The method of claim 4 wherein the fluorophore is selected from the group consisting of FAM, TET, JOE, HEX, TAMRA, ROX, Cy3, Cy5, and Cy5.5; and the quencher is selected from the group consisting of BHQ-1, BHQ-2, and BHQ-3.

6. The method of claim 1 wherein the primer in the quenched state is incorporated into a strand of a nucleic acid by a polymerase.

7. The method of claim 4 wherein a primer comprises a second fluorophore that is a fluorescence resonance energy transfer (FRET) acceptor compatible with a fluorophore of the probe comprising a fluorophore.

8. The method of claim 1 wherein the probe or the primer in the quenched state:
a) consists of one oligonucleotide comprising a fluorophore and a quencher; or
b) consists of a first oligonucleotide comprising a fluorophore and a second oligonucleotide comprising a quencher, wherein the first oligonucleotide is hybridized to the second oligonucleotide.

9. The method of claim 1 wherein a primer is a stem-loop primer or a double-stranded linear primer or wherein a probe is a stem-loop probe or a double-stranded linear probe.

10. The method of claim 1 wherein a primer is an allele-specific primer.

11. The method of claim 1 wherein a primer is an allele-specific primer for a BRAF mutation that encodes a B-Raf protein comprising the amino acid substitution V600E, V600K, and/or V600D.

12. The method of claim 1 further comprising extending a primer with a polymerase and a nucleotide.

13. The method of claim 1 further comprising performing a polymerase chain reaction or a real-time polymerase chain reaction.

14. The method of claim 1 further comprising:
3) contacting the sample comprising the nucleic acid with a second primer in a quenched state or with a second primer; and
4) detecting:
a) a second signal from the second primer in a detectable state if:
i) the second primer hybridizes to the nucleic acid; or
ii) the second primer hybridizes to the nucleic acid and the second primer is incorporated into an amplicon; or
b) a second signal from the second primer if the probe hybridizes to a complement of the nucleic acid comprising the second primer,
wherein the nucleic acid comprises a BRAF gene or a portion of a BRAF gene and the nucleic acid is detected in the sample when the second signal is detected.

15. A kit for detecting a BRAF allele comprising:
1) a detection reagent for detecting one or more BRAF alleles, wherein the detection reagent comprises one of the following:
a) a stem-loop primer comprising a fluorophore and a quencher;
b) a double-stranded linear primer comprising an allele-specific single-stranded primer comprising a fluorophore and a complementary quenching oligonucleotide;
c) a double-stranded linear primer comprising an allele-specific single-stranded primer comprising a fluorophore and a complementary oligonucleotide;
d) an allele-specific primer comprising a fluorophore and a double-stranded probe comprising a probe strand comprising a second fluorophore and a quencher oligonucleotide;
e) an allele-specific primer comprising a fluorophore and a double-stranded probe comprising a probe strand comprising a second fluorophore;
f) an allele-specific primer comprising a fluorophore and a single-stranded probe comprising a second fluorophore; or
g) an allele-specific primer comprising a fluorophore and a stem-loop probe comprising a second fluorophore and a quencher; and
2) a control nucleic acid comprising a nucleotide sequence from a BRAF gene or from a portion of a BRAF gene.

16. The kit of claim 15 further comprising:
2) a second detection reagent for detecting a second BRAF allele, wherein the second detection reagent comprises:
a) a second stem-loop primer comprising a second fluorophore and the quencher or a second quencher;
b) a second double-stranded linear primer comprising a second allele-specific single-stranded primer comprising a second fluorophore and the complementary quenching oligonucleotide or a second complementary quenching oligonucleotide; or
c) a second allele-specific primer comprising a third fluorophore.

17. The kit of claim 16(c) further comprising:
i) a second double-stranded probe comprising a second probe strand comprising a fourth fluorophore and the quencher oligonucleotide;
ii) a second double-stranded probe comprising a second probe strand comprising a fourth fluorophore and a second quencher oligonucleotide;
iii) a second stem-loop probe comprising a fourth fluorophore and the quencher;
iv) a second stem-loop probe comprising a fourth fluorophore and a second quencher; or
v) a second single-stranded probe comprising a fourth fluorophore.

18. The kit of claim 15 the fluorophore and the second fluorophore are a FRET pair.

19. The kit of claim 15 wherein the BRAF allele comprises a mutation that encodes a B-Raf protein comprising an amino acid substitution that is V600E, V600K, and/or V600D.
